(19) United States
(12) Patent Application Publication
(10) Pub. No.: US 2022/0339370 A1
(43) Pub. Date: Oct. 27, 2022

(54) NEEDLE ASSEMBLY HAVING NEEDLE SHIELD AND PLUG

(71) Applicant: Novo Nordisk A/S, Bagsvaerd (DK)

(72) Inventor: Henrik Bengtsson, Taastrup (DK)

(73) Assignees: Novo Nordisk A/S, Bagsvaerd (DK); Novo Nordisk A/S, Bagsvaerd (DK)

(21) Appl. No.: 17/760,565

(22) PCT Filed: Sep. 22, 2020
(86) PCT No.: PCT/EP2020/076423

§ 371 Date: Mar. 15, 2022

(30) Foreign Application Priority Data
Sep. 24, 2019 (EP) 19199276.7
Nov. 22, 2019 (EP) 19210845.4
Dec. 20, 2019 (EP) 19218778.9

(51) Int. Cl. A61M 5/32; A61M 5/20; A61M 5/24

(52) U.S. Cl. CPC A61M 5/3272; A61M 5/326; A61M 5/3204; A61M 5/2053; A61M 5/2466; A61M 5/2033; A61M 2205/0205; A61M 2005/311

(57) ABSTRACT

The present invention provides a needle assembly (1) for a drug delivery device, comprising a needle hub (25) in which a needle (15) is fixedly mounted, the needle (15) extending along a reference axis and comprising a needle body with a lumen and a distal needle end portion adapted for insertion through a skin layer, a needle shield (12) axially displaceable relative to the needle hub (25) between an extended position in which the distal needle end portion is covered and a retracted position in which the distal needle end portion is exposed, the nee-dle shield (12) being biased towards the extended position, and an elastomeric plug member (10) fitted tightly around a portion of the needle (15), the plug member (10) comprising a self-sealing front section (10.4) and being axially displaceable along the needle body be-tween a proximal plug position in which the distal needle end portion is exposed and a distal plug position in which the distal needle end portion is covered and the lumen is sealed by the self-sealing front section (10.4), wherein the needle shield (12) and the plug member (10) comprise mutually interactable engagement members (12.1, 12.4, 10.5) configured to ensure displacement of the plug member (10) from the proximal plug position to the distal plug position in response to a displacement of the needle shield (12) from the retracted posi-tion to the extended position.

### FIELD OF THE INVENTION

The present invention relates to needle assemblies for drug injection devices.

### BACKGROUND OF THE INVENTION

Parenteral drug administration carried out using a traditional vial and syringe system is increasingly being substituted by administration using a pen injection device. Pen injection devices are particularly convenient in that they allow the user to perform a dosed injection from a prefilled drug reservoir without first having to manually transfer the particular dose from one reservoir (the vial) to another (the syringe).

Predominantly, two types of pen injection devices are available, durable injection devices being capable of delivering one or more doses of drug from a prefilled drug cartridge which can be loaded into the device before use and replaced after exhaustion, and disposable injection devices being capable of delivering one or more doses of drug from a prefilled and non-exchangeable drug cartridge. Each of these types of pen injection devices are, or may in principle be, realised in various sub-types, such as e.g. single shot devices adapted to deliver only one dose of a predetermined, or selected, size from a drug cartridge, multi-shot devices capable of delivering a plurality of doses from a drug cartridge, manual devices, where the user provides the force needed for injection, automatic devices having a built-in energy source releasable to occasion the injection, fixed dose devices adapted to deliver doses of identical size, variable dose devices offering delivery of a plurality of doses of drug, each settable by the user from a range of possible dose sizes, etc.

As the labels suggest a durable injection device is intended for use over a considerable period of time during which multiple drug cartridges are exhausted and replaced, whereas a disposable injection device is intended for use until its dedicated drug cartridge is exhausted, after which the entire injection device is discarded.

Pen injection devices are conventionally used with matching pen needle assemblies which provide access to a subcutaneous compartment and serve as a means for administration of the drug thereto. However, many people dislike the thought of having an injection needle inserted through the skin. An undisclosed number of people even suffer from needle-phobia, and these people often benefit from using needle units with shielded needles, where the injection needle remains out of sight during handling of the needle unit, including insertion of the injection needle into the skin.

Typically, this type of needle unit comprises an axially movable sheath which can be slid between a first position in which it covers the injection needle and a second position in which the injection needle is exposed and ready for injection. In some cases, the sheath is spring loaded such that it is automatically slid back to the first position when the injection needle is retracted from the skin. An example of this is disclosed in US 2003/0078546 (Jensen).

Such automatically returned sheaths have the further advantage of automatically covering the needle tip once the needle is retracted from the skin, thereby reducing the risk of accidental needle stick injuries. They do not, however, prevent dripping from the needle which may occur if the needle is removed from the skin before the dose expelling mechanism, especially the cartridge piston, has undergone complete relaxation.

It sometimes happens that a person experiences discomfort during an injection, for example due to pressure build-up in the skin from administration of a large liquid volume or due to the injection being performed too close to a nerve or a previous injection site. In such a case it is convenient if the injection can be paused to allow removal of the injection needle and reinsertion at a different site for completion of the dose delivery. Otherwise, the user must either accept the feeling of discomfort during the entire dose administration or accept that a volume of the drug is lost to the surroundings when the needle is pulled out of the skin. Neither is desirable, the latter especially not if the drug is expensive or difficult to obtain.

Some automatic injection devices offer the possibility of pausing an ongoing injection. This involves stopping the advancement of the piston inside the cartridge by halting the piston actuation mechanism. A halting of the piston actuation mechanism is, however, not immediately possible with simple actuator solutions such as those typically preferred in connection with disposable single shot devices to keep the manufacturing costs at an acceptable level. It is therefore desirable to provide a solution which allows a user of an automatic injection device to pause an injection regardless of whether the injection device employs a complicated and expensive piston actuation mechanism or not.

### SUMMARY OF THE INVENTION

It is an object of the invention to eliminate or reduce at least one drawback of the prior art, or to provide a useful alternative to prior art solutions.

In particular, it is an object of the invention to provide a solution which prevents an injection needle from dripping following removal from the skin.

It is a further object of the invention to provide a relatively simple and inexpensive solution whereby an injection procedure can be paused, with no or only insignificant loss of drug, to allow repositioning of the injection needle.

In the disclosure of the present invention, aspects and embodiments will be described which will address one or more of the above objects and/or which will address objects apparent from the following text.

In one aspect the invention provides a needle assembly according to claim 1.

Accordingly, a needle assembly for a drug delivery device is provided. The needle assembly comprises a) a needle hub carrying a fixedly mounted needle which extends along a reference axis and comprises a needle body with a lumen and a distal needle end portion adapted for insertion through a skin layer, b) a needle shield which is axially displaceable relative to the needle hub between an extended position, in which the distal needle end portion is covered, and a retracted position, in which the distal needle end portion is exposed, and which is biased towards the extended position, and c) an elastomeric plug member which is fitted tightly around a portion of the needle. The plug member comprises a self-sealing front section and is axially displaceable along the needle body between a proximal plug position in which the distal needle end portion is exposed and a distal plug position in which the distal needle end portion is covered and the lumen is sealed by the self-sealing front section. The needle shield and the plug member comprise mutually interactable engagement members configured to ensure displacement of the plug member from the proximal plug position to the distal plug position in response to a displacement of the needle shield from the retracted position to the extended position.

The needle assembly may further comprise attachment means for either releasable or non-releasable attachment to a drug expelling unit to thereby form the drug delivery device. Alternatively, the needle assembly and the drug expelling unit are formed as a unitary device.

This solution ensures that once the needle has been inserted into the skin and an injection has been started from the drug delivery device a removal of the needle from the skin, e.g. immediately after the drug delivery device issues an end-of-dose confirmation, will cause the needle shield to automatically return to the extended position while dragging the plug member to the distal plug position, where the distal needle end portion is covered, and the flow way thus is blocked. No dripping can accordingly occur. Furthermore, since the distal needle end portion is enveloped in the plug member, any remaining risk of accidental needle stick injuries following the movement of the needle shield to the extended position are eliminated.

The mutually interactable engagement members may further be configured to ensure displacement of the plug member from the distal plug position to the proximal plug position in response to a subsequent movement of the needle shield from the extended position to the retracted position.

Thereby, the plug member will follow subsequent movements of the needle shield and allow for further use of the drug delivery device. For example, should a user for some reason wish to change injection site during an injection a removal of the needle from the skin will cause an automatic displacement of the plug member to the distal plug position, as explained above, whereby the flow way will be blocked and the dose expelling accordingly interrupted. Notably, this happens without the need for mechanical interference with an actuation mechanism in the drug delivery device.

At subsequent insertion of the needle into a different injection site the plug member is displaced to the proximal plug position as the needle shield is displaced to the retracted position when the drug delivery device is pressed against the skin surface. The distal needle end portion thereby penetrates the self-sealing front section to re-open the flow way and allow further expelling of the liquid drug. If the drug delivery device is an automatic device the dose expelling will continue immediately upon the distal needle end portion penetrating the self-sealing front section.

If the mutually interactable engagement members are configured to axially interlock the needle shield and the plug member, e.g. upon a first movement of the needle shield from the extended position to the retracted position, any axial movement of the needle shield, or any axial movement of the needle shield subsequent to the first movement from the extended position to the retracted position, will be performed jointly with the plug member, which means that in case of a multi-shot type of device a pausing of all injections is possible.

The needle assembly may further comprise a track sleeve at least partially surrounding the needle shield and the plug member and defining a plurality of tracks comprising an axially extending track and a circumferentially extending track. In that case the needle shield comprises a radial protrusion for sliding accommodation in the axially extending track and the plug member comprises a radially protruding plug arm for sliding accommodation in the circumferentially extending track. Furthermore, the circumferentially extending track comprises an axial track segment which the plug arm travels during displacement of the plug member from the proximal plug position to the distal plug position.

The track sleeve will enable easy definition of the movement pattern of the needle shield and the plug member, as the radial protrusion and the plug arm then function as respective track followers.

The track sleeve may be rotatably arranged with respect to the needle hub and the plug member may be rotationally fixed with respect to the needle hub. In that case the circumferentially extending track further comprises a helical track segment being connected to the axial track segment, and the plug member is initially axially displaceable from the distal plug position to the proximal plug position by the plug arm travelling the helical track segment in response to a rotation of the track sleeve relative to the needle hub.

This allows for an initial displacement of the plug member from the distal plug position to the proximal plug position, independent of the needle shield, by rotation of the track sleeve relative to the needle hub. The needle assembly can thereby be delivered by the manufacturer in a state where the distal needle end portion is fully enveloped, and the risk of needle stick injuries is eliminated, yet where an initial check of the condition of the needle tip is possible before performing the very first injection. Furthermore, the user will not have to overcome the friction force between the needle and the tightly fitted plug member during insertion of the needle into the skin, as the plug member is then already in the proximal plug position.

The mutually interactable engagement members may be adapted to engage when the plug member has reached the proximal plug position following displacement along the helical track segment.

In exemplary embodiments of the invention the mutually interactable engagement members comprise the plug arm and a shield hook arranged at an end portion of a deflectable proximal extension of the needle shield, where the shield hook is adapted to pass and snap behind the plug arm during movement of the needle shield from the extended position to the retracted position when the plug member is in the proximal plug position.

The track sleeve may comprise two axially and rotationally interlocked sleeve parts, where one of the two sleeve parts comprises a first helical surface defining a first portion of the helical track segment and the other of the two sleeve parts comprises a second helical surface defining a second portion of helical track segment.

The helical track segment is then formed when the two sleeve parts are interlocked during manufacturing. An arrangement with two separate interconnectible sleeve parts improves the assembly process because it provides for easy fitting of the plug member in the track sleeve.

The circumferentially extending track may further comprise an initial circumferential track segment leading into a distal end portion of the helical track segment, and the initial circumferential track segment may comprise a constriction of smaller dimension than a radial dimension of the plug arm.

Such an initial circumferential track segment will allow for a locked initial state of the needle assembly in that the plug member is then prevented from axial displacement until the plug arm passes the constriction and reaches the helical track segment, and the plug arm only passes the constriction if a torque of a predetermined size is applied to the track sleeve to induce a relative rotation between the initial circumferential track segment and the plug arm, the latter being rotationally fixed relative to the needle hub. The constriction may be dimensioned such that the size of the torque required to thus lead the plug arm therethrough is greater than the torque required to subsequently lead the plug arm along the helical track section.

The circumferentially extending track may further comprise an end track segment extending circumferentially from a distal end portion of the axial track segment, and the end track segment may comprise a non-return geometry enabling one-way motion of the plug arm from the axial track segment into the end track segment.

This is relevant in case the needle assembly is intended for a single use, as the user can apply a torque to the track sleeve following a finalised injection and thereby force the plug arm into the end track segment past the non-return geometry. When the plug arm is thus trapped in the end track segment the plug member is prevented from displacement back to the proximal plug position, even if a proximally directed force is applied to the needle shield. If the needle shield and the plug member are axially interlocked the needle shield is thereby also prevented from undergoing proximal displacement, and the needle assembly is thus turned into a sharps container.

The circumferentially extending track may further comprise a second axial track segment and a second helical track segment being interconnected and respectively connected with the helical track segment and the axial track segment so as to form an uninterrupted track configuration extending 360° along the track sleeve.

The needle assembly is thereby adapted for multiple use by repeated rotation of the track sleeve about the needle hub, where a 360° rotation of the track sleeve provides for two injection events, and where the plug member is displaced independently of the needle shield before each injection. The needle assembly may further comprise a decoupling mechanism configured to decouple the needle shield and the plug member in response to a rotation of the track sleeve relative to the needle hub.

The plug member may be a biostatic component further comprising a micro-bacterial growth inhibitor. In particular, the plug member may be made of a thermoplastic elastomer containing immobilised Zinc (Zi++) or immobilised silver (Ag++), as these ions are known to inhibit micro-bacterial growth. Since the plug member fits tightly around a portion of the needle any micro-bacterial contaminants thereon will resultantly be neutralised. The needle is thereby kept in a biostatic environment between injections and the needle assembly is accordingly suitable for multiple use.

The plug member may further comprise a cylindrical plug body defining a solid plug portion with a cylindrical channel therein. In that case the cylindrical channel abuts the self-sealing front section and has a transversal channel dimension which is smaller than a transversal dimension of the needle body.

Even though the cylindrical channel has a smaller transversal dimension than the needle and thus exerts a radial pressure on the needle body its presence reduces the frictional forces between the needle body and the plug member compared to a situation where the plug member does not comprise a channel and the needle must transpierce a solid plug body. The bias force on the needle shield can thus be smaller when the plug member comprises a pre-fabricated void for reception of the needle. If the bias force is provided by a spring member this means that the spring member can be less powerful and thereby less expensive, which is particularly relevant for disposable drug delivery devices.

In a second aspect the invention provides a drug delivery device comprising a drug expelling unit and a needle assembly as described above. The drug expelling unit comprises i) a drug reservoir holder adapted to accommodate a variable volume drug reservoir, and ii) a dose expelling mechanism for pressurising the variable volume drug reservoir.

Accordingly, a needle assembly as described above in combination with a drug expelling unit may be provided, comprising: a drug reservoir holder adapted to accommodate a variable volume drug reservoir in a position where a proximal needle end portion of the needle is fluidly connected with a liquid substance in an interior of the variable volume drug reservoir, and a dose expelling mechanism configured to expel a dose of the liquid substance through the needle, the dose expelling mechanism comprising a power source activatable to release stored energy to cause a pressurisation of the variable volume drug reservoir.

The drug reservoir holder may hold a variable volume drug reservoir when the drug expelling unit is offered by the manufacturer, or it may be adapted to receive a variable volume drug reservoir inserted by a user.

The drug expelling unit may further comprise a dose release member comprising a distal abutment surface adapted to cooperate with a proximal abutment surface of the needle shield and an axially extending leg member displaceable by means of the proximal abutment surface from a distal leg member position to a proximal leg member position to activate the power source.

The dose expelling mechanism is thus needle shield triggered and will automatically initiate a dose expelling in response to a displacement of the needle shield from the extended position to the retracted position, thereby providing a drug delivery device with a very simple user interface and handling pattern.

In exemplary embodiments of the invention the proximal abutment surface forms part of the shield hook.

The variable volume drug reservoir may be a cartridge type container being sealed distally by a penetrable self-sealing septum and proximally by an axially advanceable piston, and the dose expelling mechanism may further comprise an axially advanceable piston rod, and the power source may comprises a pre-tensioned compression spring being releasable, by displacement of the axially extending leg member from the distal leg member position to the proximal leg member position, to apply a driving force to the piston rod.

For the avoidance of any doubt, in the present context the term “drug” designates a medium which is used in the treatment, prevention or diagnosis of a condition, i.e. including a medium having a therapeutic or metabolic effect in the body. Further, the terms “distal” and “proximal” denote positions at, or directions along, a drug delivery device, a drug reservoir, or a needle unit, where “distal” refers to the drug outlet end and “proximal” refers to the end opposite the drug outlet end.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 is an exploded view of a needle assembly according to an exemplary embodiment of the invention,

FIG. 2 is an exploded view of an injection unit used in combination with the needle assembly of FIG. 1,

FIGS. 3-8 are different views of various components of the needle assembly,

FIGS. 9-14 are different views of various components of the injection unit,

FIG. 15 is a partially sectioned perspective view of an injection device according to an exemplary embodiment of the invention formed by combining the needle assembly and the injection unit,

FIG. 16 is a partially sectioned side view of the injection device in a pre-injection state,

FIG. 17 is a two-dimensional representation of track configurations in the needle assembly,

FIG. 18 is a two-dimensional representation of plug and needle shield movements in the track configurations during use of the injection device, and

FIG. 19 illustrates the injection device at various stages during an injection procedure.

In the figures like structures are mainly identified by like reference numerals.

### DESCRIPTION OF EXEMPLARY EMBODIMENTS

When/If relative expressions, such as “upper” and “lower”, “left” and “right”, “horizontal” and “vertical”, “clockwise” and “counter-clockwise”, etc., are used in the following, these refer to the appended figures and not necessarily to an actual situation of use. The shown figures are schematic representations for which reason the configuration of the different structures as well as their relative dimensions are intended to serve illustrative purposes only.

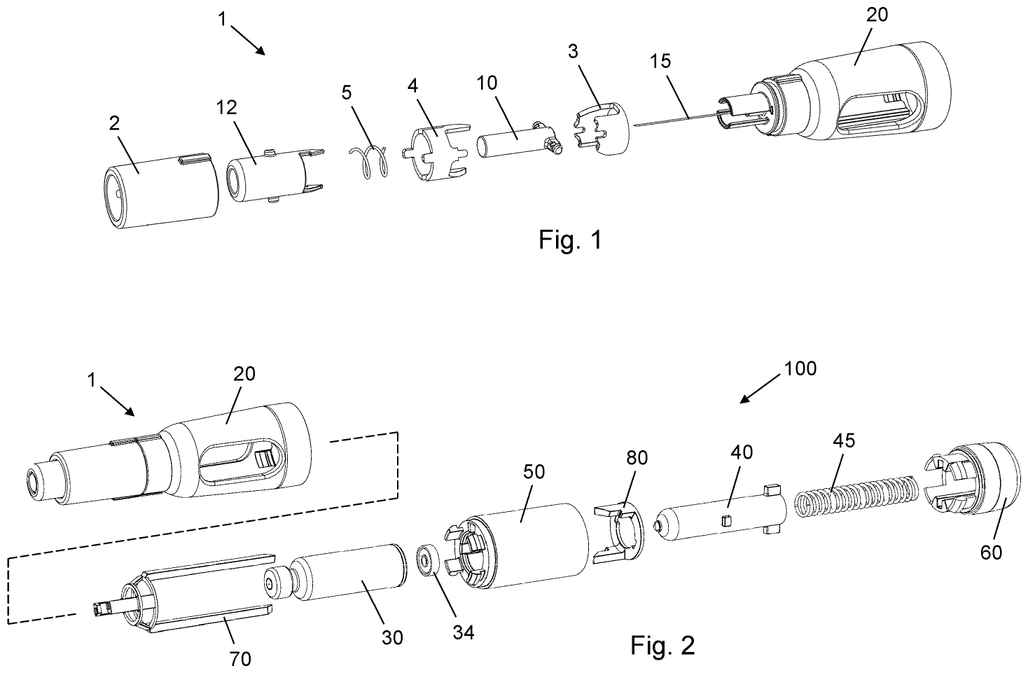

FIG. 1 is an exploded view of a needle assembly 1 according to an exemplary embodiment of the invention. The needle assembly 1 comprises a cartridge holder 20 in which an injection needle 15 is fixedly mounted, a track sleeve assembly in the form of an inner track sleeve 3, an intermediate track sleeve 4, and an outer track sleeve 2, a needle shield 12, a shield spring 5 in the form of a small compression spring, and a plug 10. The injection needle 15 is a straight tubing extending along a reference axis, and the plug 10 is biostatic in that it is made of a thermoplastic elastomer containing immobilised Zinc (Zi⁺⁺) for neutralising micro-bacterial contaminants. The inner track sleeve 3, the intermediate track sleeve 4, and the outer track sleeve 2 are both axially and rotationally interlocked, functioning as a single unit.

FIG. 2 is an exploded view of an injection unit which together with the needle assembly 1 forms an injection device 100. The injection unit comprises a cartridge 30, a piston washer 34, and a dose release member 70, accommodated in the cartridge holder 20, and a central housing part 50 with an end housing part 60 attached thereto accommodating a piston rod 40, a drive spring 45 in the form of a pretensioned compression spring, and a dose release disc 80.

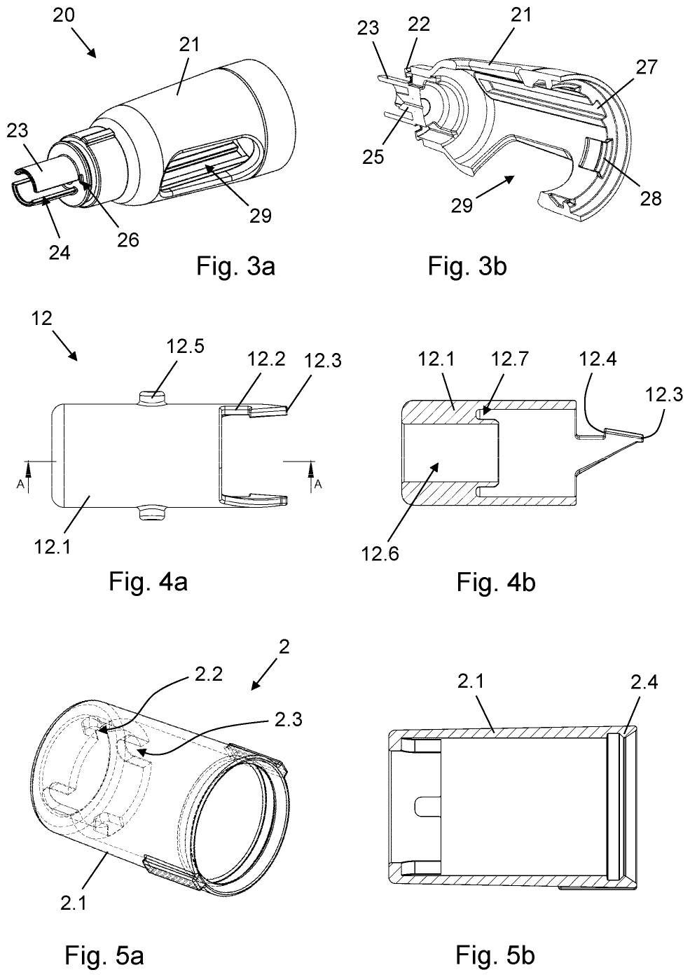

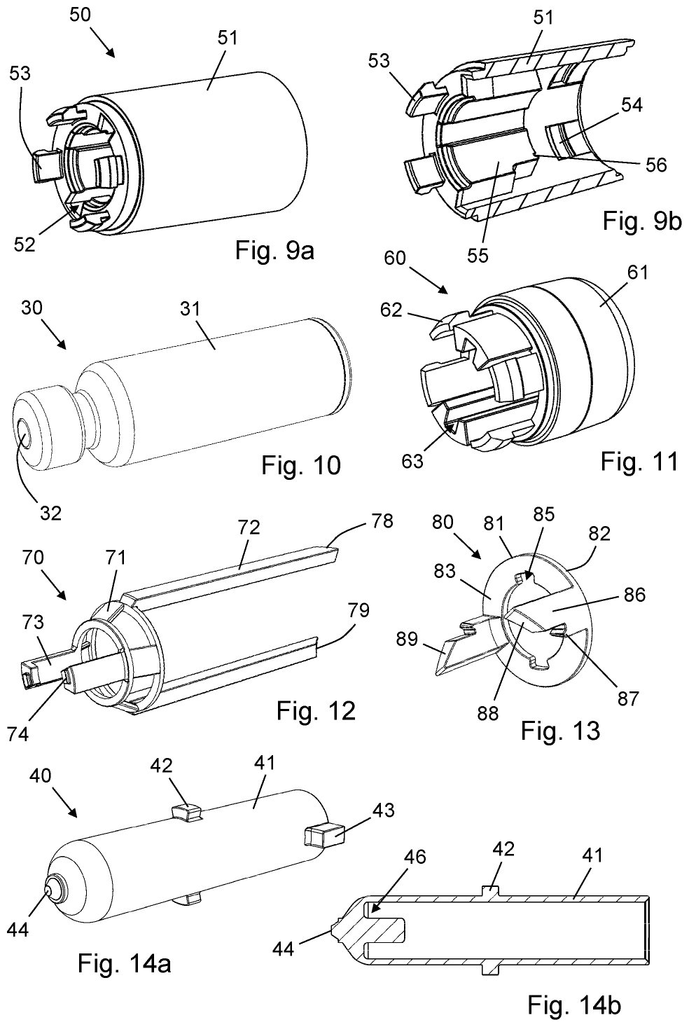

In the following details of selected components of the injection device 100 will be discussed, referring to FIGS. 3-8 for components of the needle assembly 1 and to FIGS. 9-14 for components of the injection unit.

FIGS. 3a and 3b are, respectively, a perspective view and a longitudinal section perspective of the cartridge holder 20, which has a tubular cartridge holder body 21 configured to receive a drug cartridge, the contents of which may be inspected through a window 29. At its distal end portion, the cartridge holder 20 has an integrated needle hub 25 to which the injection needle 15 (not shown) is glued. The needle hub 25 comprises a transversal surface from which a pair of opposite plug guides 23 extend distally, forming axial guiding slots 24, and in which a pair of through-going bores 26 are provided (only one is visible). The periphery of the transversal surface forms a circumferential flange 22 which functions as attachment means for axial fixation of the outer track sleeve 2 to the cartridge holder 20. Two axial tracks 27 (only one is visible) are formed along interior surface portions of the cartridge holder body 21 and serve as guides for movements of the dose release member 70. Also, four indentations 28 (only one is fully visible) are provided allowing for attachment of the cartridge holder 20 to the central housing part 50. It is noted that the fact that the needle hub 25 holding the injection needle 15 in the present embodiment forms an integral part of the cartridge holder 20 is irrelevant to the exercise of the invention and that the needle hub alternatively could be a separate component being releasably or non-releasably attachable to the cartridge holder 20.

FIGS. 4a and 4b are, respectively, a side view and a longitudinal section view of the needle shield 12 which has a generally cylindrical shield body 12.1 with proximally extending opposite fingers 12.2, each finger 12.2 having a harpoon-like end configuration with a shield tip 12.3 for interaction with the dose release member 70 and a shield hook 12.4 for interaction with the plug 10. A pair of opposite protrusions 12.5 are provided on an exterior wall of the shield body 12.1. Furthermore, an interior portion of the shield body 12.1 is formed with a wall thickening to provide an interior chamber 12.6 for sliding reception of the plug 10 as well as a shield spring socket 12.7 for support of a distal end portion of the shield spring 5.

FIGS. 5a and 5b are, respectively, a perspective view and a longitudinal section view of the outer track sleeve 2 which has a generally cylindrical sleeve wall 2.1 of larger dimension than the shield body 12.1. In FIG. 5a an interior portion of the sleeve wall 2.1 is indicated with dotted lines to provide a three-dimensional visualisation of inner geometries which include a pair of opposite female coupling portions 2.2 for interaction with the intermediate track sleeve 4 and a pair of opposite distal shield track portions 2.3 each configured for sliding reception of one of the protrusions 12.5 on the shield body 12.1. An interior rim 2.4 is provided at a proximal end portion of the sleeve wall 2.1 for snap engagement with the circumferential flange 22.

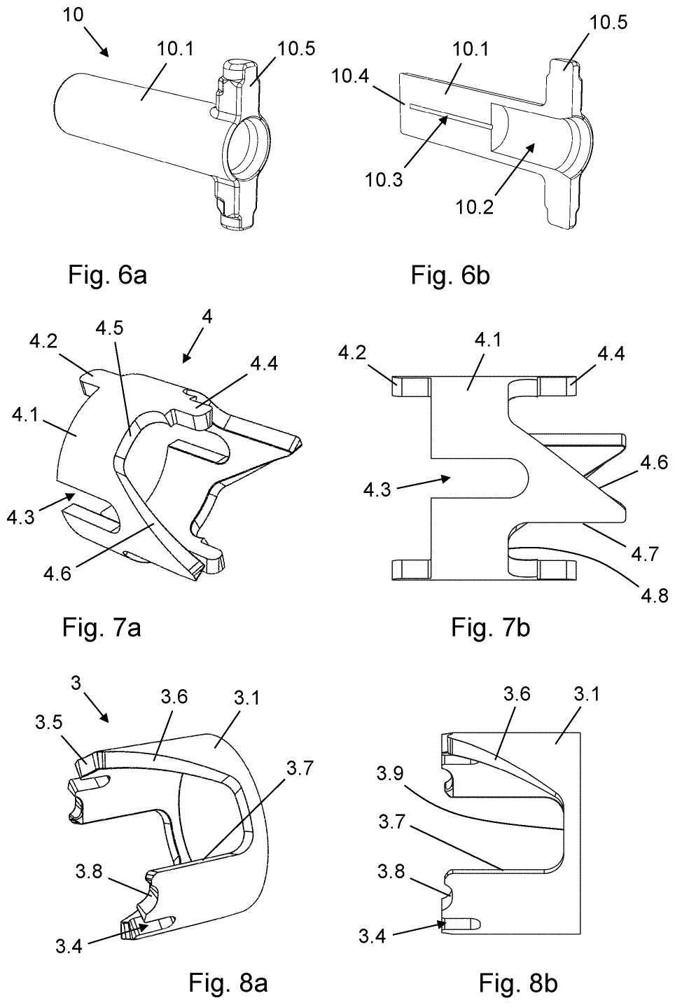

FIGS. 6a and 6b are, respectively, a perspective view and a longitudinal section perspective of the plug 10 which has a generally cylindrical plug body 10.1 with a proximal receiving chamber 10.2 capable of accommodating the needle hub 25 and a distal cylindrical channel 10.3 of a diameter slightly smaller than the transversal dimension of the injection needle 15 to ensure a fluid tight slidable reception and accommodation thereof. The cylindrical channel 10.3 is sealed distally by a self-sealing front section 10.4 which the injection needle 15 is able to penetrate. At its proximal end portion, the plug 10 is provided with a pair of transversally extending plug arms 10.5 adapted for sliding movement in the guiding slots 24 and for interaction with respective interior geometries of the inner track sleeve 3 and the intermediate track sleeve 4, which are configured to provoke a movement of the plug arms 10.5 in the guiding slots 24 in a manner that will be clear from the below.

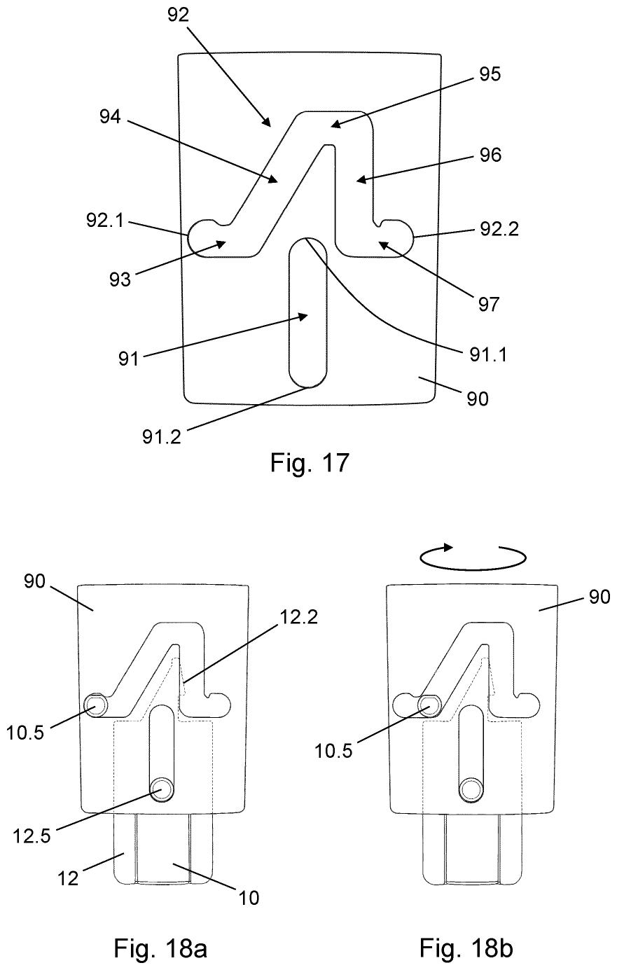

FIGS. 7a and 7b are, respectively, a perspective view and a side view of the intermediate track sleeve 4 which has an intermediate track sleeve body 4.1 with a pair of distally pointed male coupling portions 4.2 for engagement with the female coupling portions 2.2, ensuring a rotationally interlocked relationship between the outer track sleeve 2 and the intermediate track sleeve 4, and a pair of proximally pointed male coupling portions 4.4 for interaction with the inner track sleeve 3. The intermediate track sleeve body 4.1 is further provided with proximal shield track portions 4.3 in the form of two opposite axial slots, respective start seats 4.5 for the plug arms 10.5, respective lower parts of helical guide surfaces 4.6 connected with the start seats 4.5, respective axial guide surfaces 4.7 connected with the helical guide surfaces 4.6, and respective end seats 4.8 connected with the axial guide surfaces 4.7. The proximal shield track portions 4.3 and the distal shield track portions 2.3 together form a pair of axial tracks for the protrusions 12.5, as shown in and described in connection with FIGS. 17-19.

FIGS. 8a and 8b are, respectively, a perspective view and a side view of the inner track sleeve 3 which has an inner track sleeve body 3.1 with a pair of female coupling portions 3.4 for engagement with the proximally pointed male coupling portions 4.4, ensuring a rotationally interlocked relationship between the intermediate track sleeve 4 and the inner track sleeve 3. The inner track sleeve body 3.1 is further provided with respective start seat tops 3.5, respective upper parts of helical guide surface 3.6, respective connecting seats 3.9, respective axial guide surfaces 3.7, and respective end seat tops 3.8, which in cooperation with the respective start seats 4.5, the respective lower parts of helical guide surfaces 4.6, the respective axial guide surfaces 4.7, and the respective end seats 4.8 provide a track configuration for determining motion of the plug arms 10.5, and thereby of the plug 10 as such. This track configuration is described in more detail in connection with FIGS. 17-19.

FIGS. 9a and 9b are, respectively, a perspective view and a longitudinal section perspective of the central housing part 50 which has a generally cylindrical housing wall 51 and four distally directed snap hooks 53 for snap fit engagement with the indentations 28, ensuring an axially and rotationally interlocked relationship between the cartridge holder 20 and the central housing part 50. Longitudinal guide tracks 52 for the dose release member 70 are provided in interior wall thickenings 55, which also hold a pair of notches 56 for releasable retention of the dose release disc 80, and four indentations 54 (only two are visible) are provided in a proximal end section of the housing wall 51 allowing for mechanical coupling of the central housing part 50 and the end housing part 60.

FIG. 10 is a perspective view of the cartridge 30 showing the exterior contours of a generally cylindrical cartridge wall 31 and a capped neck with a penetrable self-sealing septum 32.

FIG. 11 is a perspective view of the end housing part 60 which has a cap shaped body 61 with four distally extending snap hooks 62 for engagement with the indentations 54 and a pair of longitudinal splines 63 for rotational fixation of the piston rod 40.

FIG. 12 is a perspective view of the dose release member 70 which has a skeletal structure 71 comprising a pair of proximally pointing legs 72 with oppositely sloped ends 78, 79 for interaction with the dose release disc 80, and a pair of distally pointing arms 73 for interaction with the fingers 12.2 via respective contact points 74. The legs 72 are configured for slidable accommodation in the guide tracks 52, which thereby rotational interlock the dose release member 70 and the central housing part 50, while allowing relative axial motion therebetween.

FIG. 13 is a perspective view of the dose release disc 80 which has a generally annular disc body 81 with opposite keyholes 85, a proximal face 82, and a distal face 83 from which a pair of disc legs 86 extend. Each disc leg 86 is provided with a stud 87 for reception in one of the notches 56 and a ramp 88, 89 for respective sliding interaction with the sloped ends 78, 79 of the legs 72.

FIGS. 14a and 14b are, respectively, a perspective view and a longitudinal section view of the piston rod 40 which has a generally cylindrical piston rod body 41 adapted to extend through the dose release disc 80 and a distal piston rod tip 44 adapted to abut the piston washer 34. At a proximal end section, the piston rod body 41 is provided with opposite fins 43 (only one is visible) for slidable accommodation in the splines 63, and at a central section the piston rod body 41 has opposite lock tabs 42 providing for initial axial positioning of the piston rod 40 relative to the central housing part 50 by abutment against the proximal face 82 of the dose release disc 80. The piston rod body 41 is hollow and is adapted to accommodate the drive spring 45, axially supporting one spring end in a spring socket 46.

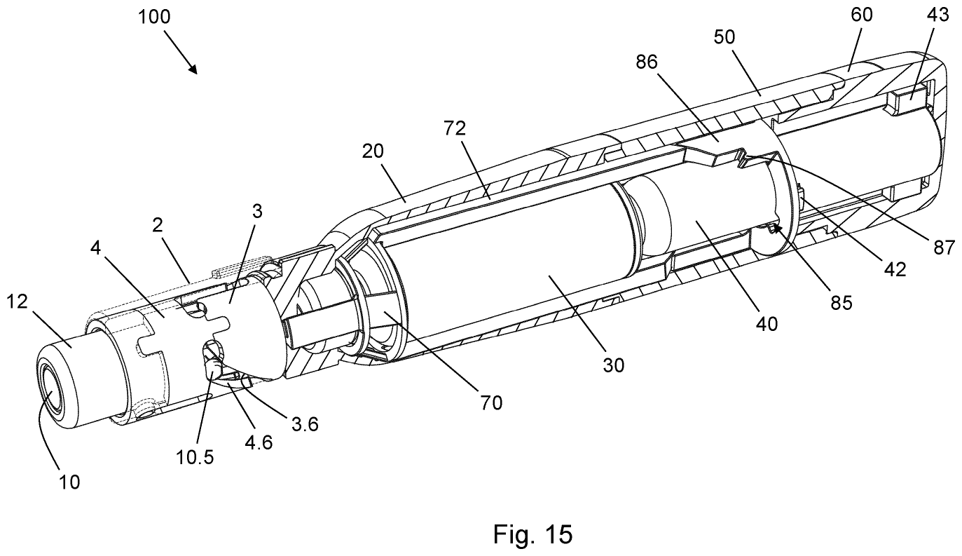

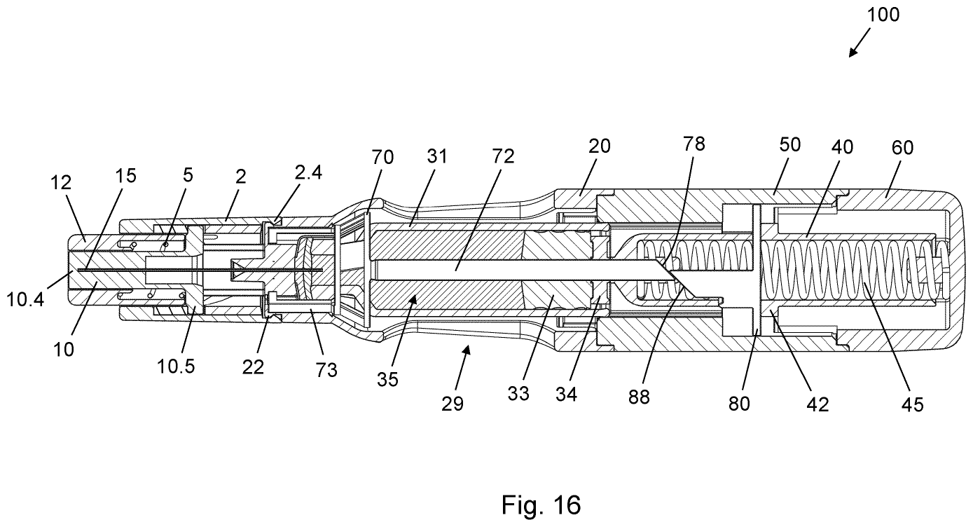

FIG. 15 shows the injection device 100 in a perspective view where the cartridge holder 20, the central housing part 50 and the end housing part 60 are longitudinally sectioned to produce an overview of the components in an assembled and ready-to-use state of the device. The outer track sleeve 2 is for the sake of clarity indicated as a see-through component with dotted lines to illustrate how the above described respective seats and guide surfaces of the inner track sleeve 3 and the intermediate track sleeve 4 form different track sections for the plug arms 10.5 to follow. As can be seen (also from FIG. 16) the outer track sleeve 2 is snap fitted to the cartridge holder 20 and thus encapsulate both the inner track sleeve 3 and the intermediate track sleeve 4 to provide the axially and rotationally interlocked track sleeve assembly. In the shown state of the injection device 100 the plug arms 10.5 rest at a track junction just before entry into a helical track section formed by the respective helical guide surfaces 3.6, 4.6. In this position of the plug arms 10.5 the plug 10 is still at its distal most position relative to the cartridge holder 20 where a distal end portion of injection needle 15 resides in the cylindrical channel 10.3 behind the self-sealing front section 10.4. The exterior surface of the self-sealing front section 10.4 is flush, or substantially flush, with a transversal end portion of the needle shield 12.

The piston rod 40 which is biased distally by the drive spring 45 acting between a proximal end surface of the end housing part 60 and the spring socket 46 is retained in the shown pre-use position by the lock tabs 42 resting on the proximal face 82 of the dose release disc 80, angularly offset from the keyholes 85.

FIG. 16 is a longitudinal section view of the injection device 100 where the dose release member 70 and the dose release disc 80 are depicted non-sectioned. The injection device 100 is in a state corresponding to the one shown n FIG. 15. It is seen that the piston rod 40 is retained in a position where the piston rod tip 44 abuts the piston washer 34 which in turn abuts a slidable piston 33 sealingly fitted to an interior portion of the cartridge wall 31. The injection needle 15 is fixedly mounted in the needle hub 25 of the cartridge holder 20 such that a rear needle portion extends through the self-sealing septum 32 and resides in a chamber of the cartridge 30 which is defined by the cartridge wall 31, the piston 33, and the self-sealing septum 32 and which holds a liquid substance 35.

FIG. 17 is a two-dimensional representation of a track sleeve assembly 90 as formed by the outer track sleeve 2, the inner track sleeve 3, and the intermediate track sleeve 4 in the manner described above. For the sake of clarity, only one of the two diametrically opposite track configurations are visualised, and in the following the details of the structure and use of track sleeve assembly 90 will be explained based on this one track configuration, it being implicit that a diametrically opposite track configuration with similar characteristics exist.

The track sleeve assembly 90 comprises a needle shield track 91, delimited by a proximal needle shield track end 91.1 and a distal needle shield track end 91.2, defining possible axial movement of the needle shield 12 relative to the injection needle 15, and a plug arm track configuration 92 comprising a plurality of pair-wise connected track sections, delimited by a plug arm track start 92.1 and a plug arm track end 92.2, defining possible axial movement of the plug 10 relative to the injection needle 15.

The plurality of pair-wise connected track sections comprises an initial circumferential track segment 93, which leads from the plug arm track start 92.1 to a helical track segment 94, an interconnecting track segment 95 which leads from the helical track segment 94 to an axial track segment 96, and an end track segment 97 which leads from the axial track segment 96 to the plug arm track end 92.2.

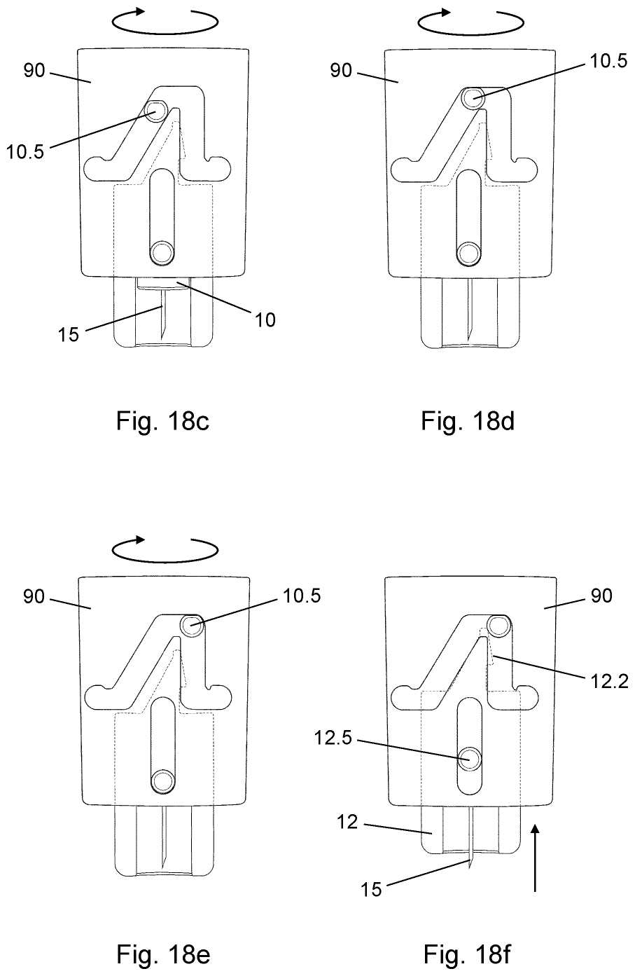

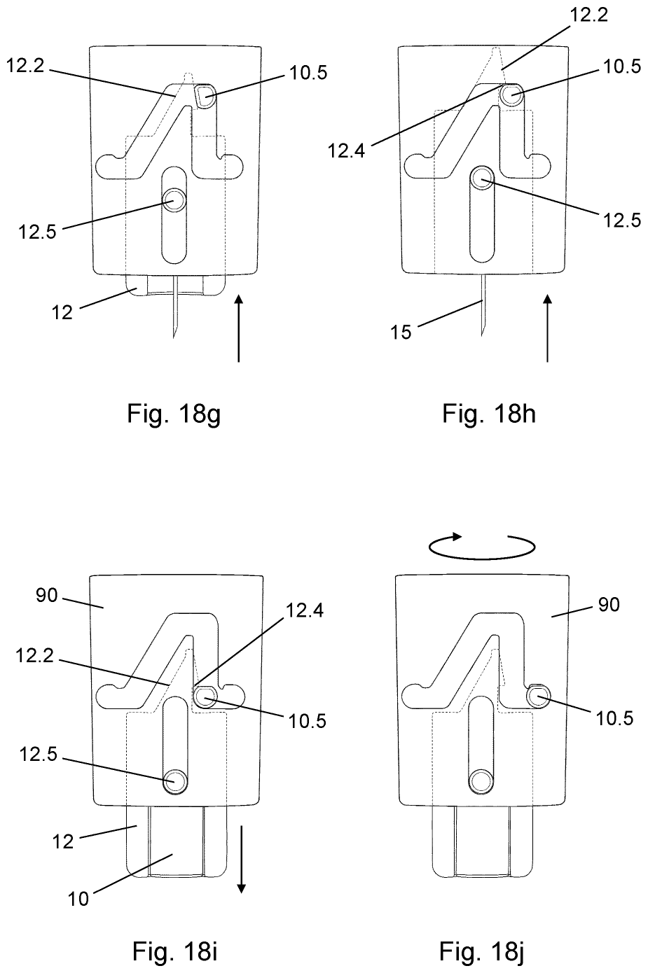

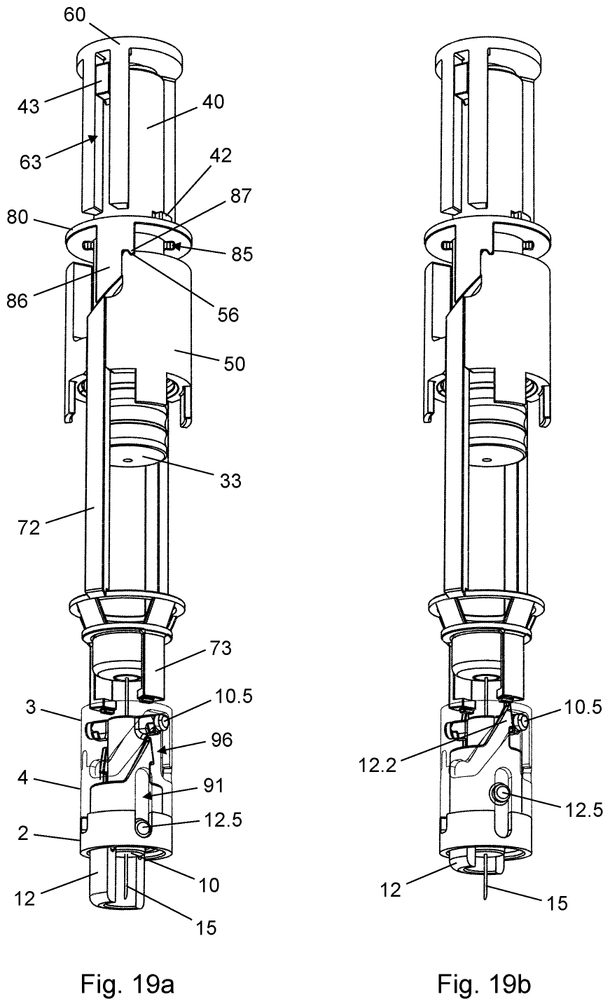

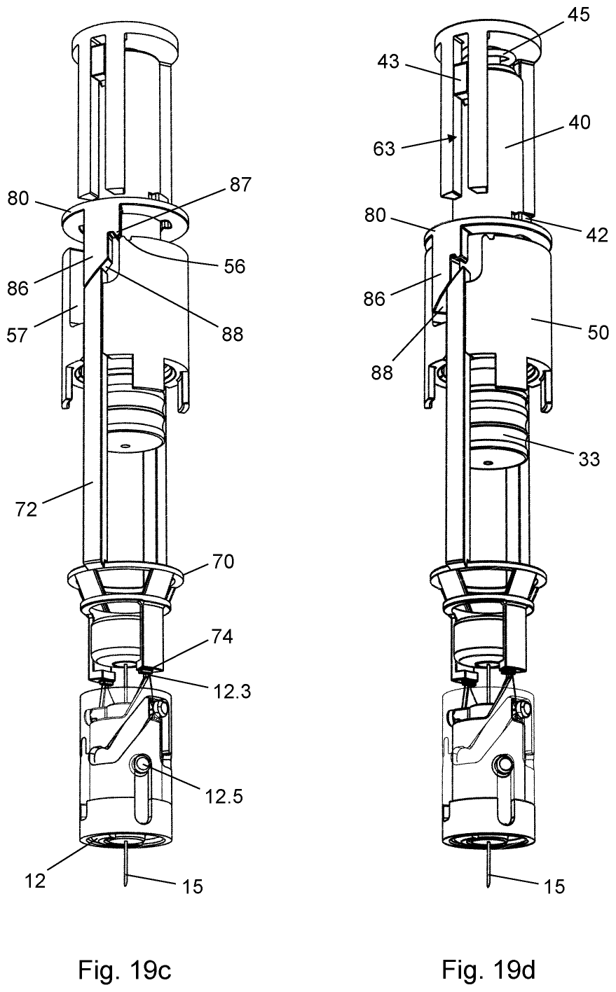

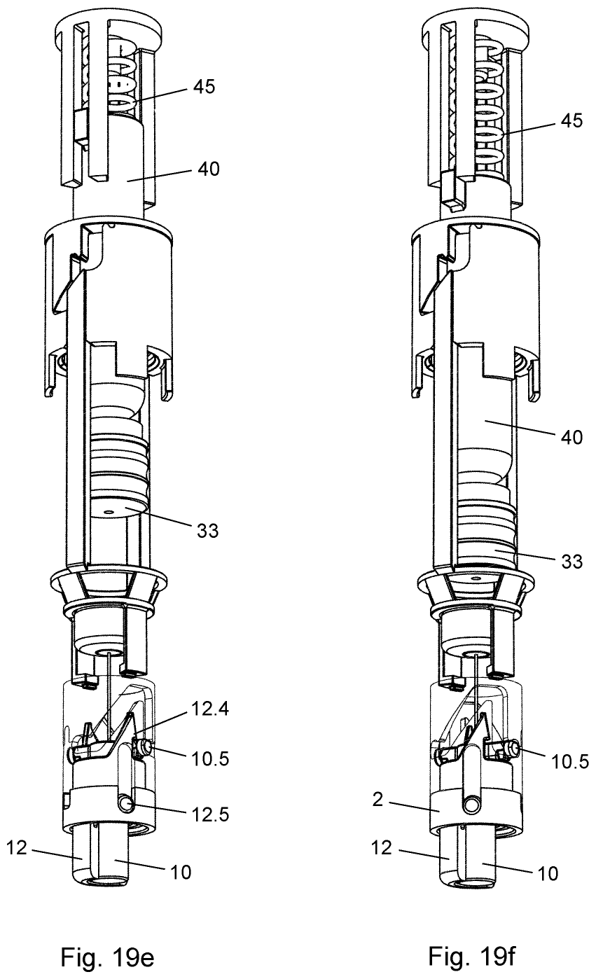

The invention will now be described in more detail with reference to FIGS. 18-19 which illustrate the movement patterns of the plug 10 and the needle shield 12 during use of the injection device 100 as well as the operating principle of the dose release mechanism.

FIG. 18a is a two-dimensional visualisation of the initial positions of the plug arms 10.5 and the needle shield 12 relative to the track sleeve assembly 90, which is fixed to the cartridge holder 20 as shown in FIG. 16, in a particular embodiment of the invention. It is noted that in other embodiments of the invention this may not represent the starting point for operation of the injection device 100. For example, the plug 10 may initially be in a retracted position relative to the track sleeve assembly 90 and the needle shield 12.

Nevertheless, in FIG. 18a the plug arms 10.5 (only one is shown, in accordance with the above) rest in the initial circumferential track segment 93 at the plug arm track start 92.1. The plug 10 is thereby in it distal most position where it covers the injection needle 15, together with the needle shield 12 which is in an extended position with the protrusions 12.5 seated at the distal needle shield track end 91.2. The portion of the needle shield 12 which is covered by the track sleeve assembly 90 is indicated with dotted lines which will be used to illustrate the interaction between the fingers 12.2 and the plug arms 10.5 at a later stage of a dose expelling action.

In order to deliver a dose from the injection device 100 the user firstly rotates the track sleeve assembly 90 clockwise (seen from a proximal perspective) relative to the cartridge holder 20. This is done e.g. by holding the cartridge holder 20 or the central housing part 50 in one hand and turning the outer track sleeve 2 with the other hand. Since the plug arms 10.5 are rotationally fixed in the guiding slots 24 the plug 10 remains immovable while the track sleeve assembly 90 is angularly displaced a short distance until the relative rotation brings the plug arms 10.5 to the beginning of the helical track segment 94. This position corresponds to the state of the injection device 100 shown in FIG. 15. The shape of the start seat top 3.5 narrows the initial circumferential track segment 93 to such a degree that the initial rotation of the outer track sleeve 2 requires a relatively high torque input and thus cannot happen inadvertently when the injection device 100 for example is moved about in a bag or a pocket.

Subsequent clockwise rotation of the outer track sleeve 2 relative to the cartridge holder 20 leads the plug arms 10.5 upwards in the helical track segment 94 and thereby causes a retraction of the plug 10 towards the needle hub 25, exposing the injection needle 15 to the immediate surroundings, albeit still within the needle shield 12. This is illustrated in FIG. 18c.

Continued clockwise rotation of the outer track sleeve 2 then leads the plug arms 10.5 into the interconnecting track segment 95, as shown in FIG. 18d, where the plug 10 is fully retracted relative to the needle hub 25, and finally on to a position at the top of the axial track segment 96, as shown in FIG. 18e.

At this point the injection device 100 is ready to deliver a dose. FIGS. 18f-18h illustrate what happens when the user places the needle shield 12 against the skin (not shown) and presses the cartridge holder 20 distally to effect an insertion of the injection needle into the subcutaneous tissue. The axial force applied by the user causes the protrusions 12.5 to travel the needle shield track 91, as the needle shield 12 is depressed into the track sleeve assembly 90 against the force from the shield spring 5. The movement of the needle shield 12 also causes the fingers 12.2 to approach the plug arms 10.5 (FIG. 180. At some point the shield hooks 12.4 reach the plug arms 10.5 and the fingers 12.2 are forced to undergo an elastic lateral deflection thereby, as indicated in FIG. 18g.

When the needle shield 12 is pressed fully into the track sleeve assembly 90 the protrusions 12.5 have reached the proximal needle shield track end 91.1, and the shield hooks 12.4 have passed the plug arms 10.5 and allowed the fingers 12.2 to return to their normal undeflected state. FIG. 18h shows how the shield hooks 12.4 now rests just proximally of the plug arms 10.5.

The movement of the needle shield 12 to a fully retracted position relative to cartridge holder 20 occasions an automatic initiation of an expelling of the liquid substance 35 from the cartridge 30, as explained below with reference to FIG. 19. As long as the user maintains the position of the injection device 100 relative to the skin where the needle shield 12 is fully depressed the drive spring 45 will provide kinetic energy to propel the piston rod 40 forward, and the liquid substance 35 will be forced through the injection needle 15 continuously in response to the resultant distal displacement of the piston 33.

If the user should desire to pause the dose expelling, e.g. to remedy a misplacement of the injection needle 15 or to change injection site to avoid pain from subdermal build-up of a large liquid volume, the injection device 100 is simply lifted off the skin surface, whereby the injection needle 15 is pulled out of the skin and the shield spring 5 automatically returns the needle shield 12 to the initial extended position. However, because the shield hooks 12.4 are now positioned behind the plug arms 10.5 the returning movement of the needle shield 12 will cause the plug 10 to be pulled distally along with the shield body 12.1, as indicated in FIG. 18i, and the distal end portion of the injection needle 15 will become re-positioned in the cylindrical channel 10.3 behind the self-sealing front section 10.4 which effectively seals off the tip of the injection needle, preventing liquid flow therethrough and consequently halting the movement of the piston 33. With this solution it is thus not necessary to provide a pause function in the dose expelling mechanism itself, which means that the injection device 100 can be made much simpler and less expensive.

When the user is ready to resume the dose delivery the needle shield 12 is simply placed against the skin at a desired injection site and the cartridge holder 20 is once more pressed distally against the force from the shield spring 5, whereby both the needle shield 12 and the plug 10 are forced proximally in the track sleeve assembly 90, the protrusions 12.5 travelling the needle shield track 91 from the distal needle shield track end 91.2 to the proximal needle shield track end 91.1 and the plug arms 10.5 travelling the axial track segment 96 back to the junction with the interconnecting track segment 95. The needle assembly 1 thereby reaches a state corresponding to the one shown in FIG. 18h, where the injection needle 15 is positioned in the subcutaneous tissue of the user and the flow path is no longer obstructed, allowing the drive spring 45 to release more energy to advance the piston rod 40 and finalise the dose expelling.

When the entire dose has been delivered from the cartridge 30 the injection device 100 is removed from the skin, whereby the shield spring 5 again urges the needle shield 12 and the plug 10 distally to cover the injection needle 15, eliminating any risk of accidental needle stick injuries. A subsequent clockwise rotation of the outer track sleeve 2 will lead the plug arms 10.5 into the end track segment 97, where they will be held firmly in position at the plug arm track end 92.2, as shown in FIG. 18j, due to the shape of the end seat top 3.8 providing a non-return geometry.

FIG. 19 shows perspective views of the injection device 100 bar a few components and component parts which have been removed to provide a clearer illustration of the dose release mechanism. Specifically, a portion of the needle shield 12 has been cut away, the outer track sleeve 2 is depicted as a see-through component, the cartridge holder 20 has been omitted entirely, except from the injection needle 15, and only respective interior portions of the central housing part 50 and the end housing part 60 are visible.

FIG. 19a illustrates the injection device 100 in a ready-to-inject state, where the plug arms 10.5 are positioned at the top of the axial track segment 96 and the plug 10 accordingly is fully retracted, exposing the distal end portion of the injection needle 15 within the needle shield 12. It is noted that in other embodiments of the invention, for example where the plug 10 is not designed to be biostatic but merely to provide a fluid tight fit around the distal end portion of the injection needle 15 when in an extended position, this could be the initial state in which the injection device 100 would be offered by the manufacturer. However, in the present embodiment at this point the outer track sleeve 2 has been rotated as described with respect to FIGS. 18a-18e in order to initially retract the plug 10. In this ready-to-inject state of the injection device 100 the drive spring 45 is cocked and the piston rod 40 is releasably retained by the dose release disc 80 being releasably fixed in the central housing part 50 due to an engagement between the studs 87 and the notches 56 and the abutment of the lock tabs 42 on the proximal face 82 applying a distal force to the disc body 81.

FIGS. 19b and 19c illustrate the proximal movement of the needle shield 12 as the injection device 100 is pressed against the skin (not shown) of the user. In FIG. 19b the protrusions 12.5 are pressed back around ⅔ the length of the needle shield track 91, and the tip of the injection needle 15 has transpierced the skin surface, while the fingers 12.2 are being deflected by the plug arms 10.5. This state corresponds to the one sketched in FIG. 18g.

In FIG. 19c, however, as the needle shield 12 becomes fully depressed the shield tips 12.3 abut the contact points 74 and displaces the dose release member 70 proximally, whereby the sloped ends 78, 79 of the legs 72 slide upwards along the ramps 88, 89 on the disc legs 86 and eventually lift the studs 87 out of the notches 56, as the dose release disc 80 also undergoes a slight counter-clockwise rotation (seen from the depicted distal perspective). This state corresponds to the one illustrated in FIG. 18h, where at the same time the shield hooks 12.4 snap in behind the plug arms 10.5.

As soon as the studs 87 leave the notches 56 the distally directed force from the drive spring 45 will force the ramps 88, 89 to slide downwards along the sloped ends 78, 79, whereby the dose release disc 80 will undergo further counter-clockwise rotation relative to the central housing part 50 until the disc legs 86 meet a stop surface 57 on the interior wall thickening 55, as shown in FIG. 19d. The rotation of the dose release disc 80 brings the keyholes 85 in angular alignment with the lock tabs 42, and the piston rod 40 is thereby released and urged distally by the drive spring 45 to advance the piston 33 in the cartridge 30 and expel a volume of the liquid substance 35 through the injection needle 15.

If the user wishes to pause the dose expelling she simply removes the injection device 100 from the skin surface. This action will cause the shield spring 5 to immediately push the needle shield 12 back to the initial extended position, and since the shield hooks 12.4 now engage the plug arms 10.5 the distal movement of the needle shield 12 will slave the plug 10 which is thus returned to the initial position relative to the track sleeve assembly 90 in which it covers the injection needle 15 and the distal end portion of the injection needle 15 is accommodated in the cylindrical channel 10.3. The fluid tight fit of the cylindrical channel 10.3 around the distal end portion of the injection needle 15 effectively seals the fluid passage and thereby prevents liquid outflow. The result is an immediate halting of the piston 33 and the piston rod 40, the two however still being biased by the drive spring 45. The injection device 100 can then be moved around with spilling any liquid substance 35. The paused state is illustrated in FIG. 19e.

When the user decides to resume the dose expelling she presses the injection device 100 against the skin once again, e.g. at a new site, whereby the needle shield 12 and the plug 10 will be depressed into the track sleeve assembly 90 together, as the biasing force from the shield spring 5 is overcome. The protrusions 12.5 thereby travel the needle shield tracks 91 from the distal needle shield track end 91.2 to the proximal needle shield track end 91.1 and the plug arms 10.5 travel the axial track segment 96 back to the top. The state of the needle assembly 1 then again corresponds to what is illustrated in FIG. 19d.

After completion of the dose expelling the user withdraws the injection needle 15 from the skin and the shield spring 5 returns the needle shield 12 and the plug 10 to the extended positions in which the injection needle 15 is covered. The outer track sleeve 2 is then rotated one final time to lead the plug arms 10.5 into the end track segment 97, in effect locking the plug 10 and the needle shield 12 in position to prevent subsequent accidental needle stick injuries.

While the present exemplary embodiment of the invention is concerned with a single shot type of injection device 100 it is noted that a slightly modified needle assembly functioning basically as set out in the above may be used in combination with a multi-shot injection device capable of delivering more than one dose of liquid substance to provide the same advantage of not requiring a pausing feature in the dose expelling mechanism. On the basis of the presently shown closed plug arm track configuration 92 such a slightly modified needle assembly could be designed with a single, circumferentially extending continuous track for the plug arms 10.5 to travel, e.g. by connecting the respective initial track segments 93 with the respective end track segments 97. Especially in such cases it would be relevant to employ a biostatic version of the plug 10 to reduce the risk of microbial contamination of the injection needle 15 between the dose expelling events.

1. A needle assembly comprising:
a needle hub in which a needle is fixedly mounted, the needle extending along a reference axis and comprising a needle body with a lumen and a distal needle end portion adapted for insertion through a skin layer,
a needle shield axially displaceable relative to the needle hub between an extended position in which the distal needle end portion is covered and a retracted position in which the distal needle end portion is exposed, the needle shield being biased towards the extended position, and
an elastomeric plug member fitted tightly around a portion of the needle, the plug member comprising a self-sealing front section and being axially displaceable along the needle body between a proximal plug position in which the distal needle end portion is exposed and a distal plug position in which the distal needle end portion is covered and the lumen is sealed by the self-sealing front section,
wherein the needle shield and the plug member comprise mutually interactable engagement members configured to ensure displacement of the plug member from the proximal plug position to the distal plug position in response to a displacement of the needle shield from the retracted position to the extended position.

2. The needle assembly according to claim 1, wherein the mutually interactable engagement members are further configured to ensure displacement of the plug member from the distal plug position to the proximal plug position in response to a subsequent movement of the needle shield from the extended position to the retracted position.

3. The needle assembly according to claim 2, further comprising a track sleeve at least partially surrounding the needle shield and the plug member and defining a plurality of tracks comprising an axially extending track and a circumferentially extending track,
wherein the needle shield comprises a radial protrusion for sliding accommodation in the axially extending track and the plug member comprises a radial plug arm for sliding accommodation in the circumferentially extending track, and
wherein the circumferentially extending track comprises an axial track segment which the radial plug arm travels during displacement of the plug member from the proximal plug position to the distal plug position.

4. The needle assembly according to claim 3, wherein the track sleeve is rotatably arranged with respect to the needle hub and the plug member is rotationally fixed with respect to the needle hub,
wherein the circumferentially extending track further comprises a helical track segment being connected to the axial track segment, and
wherein the plug member is initially axially displaceable from the distal plug position to the proximal plug position by the radial plug arm travelling the helical track segment in response to a rotation of the track sleeve relative to the needle hub.

5. The needle assembly according to claim 4, wherein the mutually interactable engagement members are adapted to engage when the plug member has reached the proximal plug position.

6. The needle assembly according to claim 4, wherein the mutually interactable engagement members comprise the radial plug arm and a shield hook arranged at an end portion of a deflectable proximal extension of the needle shield, the shield hook being adapted to pass and snap behind the radial plug arm during movement of the needle shield from the extended position to the retracted position when the plug member is in the proximal plug position.

7. The needle assembly according to claim 4, wherein the track sleeve comprises two axially and rotationally interlocked sleeve parts, and
wherein one sleeve part comprises a first helical surface defining a first portion of the helical track segment and the other sleeve part comprises a second helical surface defining a second portion of helical track segment.

8. The needle assembly according to claim 4, wherein the circumferentially extending track further comprises an initial circumferential track segment leading into a distal end portion of the helical track segment, the initial circumferential track segment comprising a constriction of smaller dimension than a radial dimension of the radial plug arm.

9. The needle assembly according to claim 4, wherein the circumferentially extending track further comprises an end track segment extending circumferentially from a distal end portion of the axial track segment, the end track segment comprising a non-return geometry enabling one-way motion of the radial plug arm from the axial track segment into the end track segment.

10. The needle assembly according to claim 4, wherein the circumferentially extending track further comprises a second axial track segment and a second helical track segment being interconnected and respectively connected with the helical track segment and the axial track segment so as to form an uninterrupted track configuration extending 360° along the track sleeve.

11. The needle assembly according to claim 1, wherein the plug member is a biostatic component further comprising a micro-bacterial growth inhibitor.

12. The needle assembly according to claim 1, wherein the plug member further comprises a cylindrical plug body through a portion of which a cylindrical channel extends, the cylindrical channel abutting the self-sealing front section and having a transversal channel dimension which is smaller than a transversal dimension of the needle body.

13. The needle assembly according to claim 1 in combination with a drug expelling unit comprising:
a drug reservoir holder adapted to accommodate a variable volume drug reservoir in a position where a proximal needle end portion of the needle is fluidly connected with a liquid substance in an interior of the variable volume drug reservoir, and
a dose expelling mechanism configured to expel a dose of the liquid substance through the needle, the dose expelling mechanism comprising a power source activatable to release stored energy to cause a pressurisation of the variable volume drug reservoir.

14. The needle assembly and drug expelling unit according to claim 13, wherein the drug expelling unit further comprises a dose release member comprising a distal abutment surface adapted to cooperate with a proximal abutment surface of the needle shield and an axially extending leg member displaceable by structure of the proximal abutment surface from a distal leg member position to a proximal leg member position to activate the power source.

15. The needle assembly and drug expelling unit according to claim 13, wherein the variable volume drug reservoir is a cartridge type container being sealed distally by a penetrable self-sealing septum and proximally by an axially advanceable piston, and
wherein the dose expelling mechanism further comprises an axially advanceable piston rod, and the power source comprises a pre-tensioned compression spring being releasable, by displacement of the axially extending leg member from the distal leg member position to the proximal leg member position, to apply a driving force to the piston rod.
